(19) United States
(12) Patent Application Publication
(10) Pub. No.: US 2026/0242330 A1
(43) Pub. Date: Aug. 20, 2026

(54) USE OF AN AMANTADINE MONONITRATE COMPOUND IN MANUFACTURE OF A MEDICAMENT FOR THE PREVENTION AND TREATMENT OF HYPERTENSION

(71) Applicant: GUANGZHOU MAGPIE PHARMACEUTICALS CO., LTD., Guangzhou (CN)

(72) Inventors: Yuqiang WANG, Guangzhou (CN); Yewei SUN, Guangzhou (CN); Gaoxiao ZHANG, Guangzhou (CN); Zaijun ZHANG, Guangzhou (CN); Peng YI, Guangzhou (CN)

(73) Assignee: GUANGZHOU MAGPIE PHARMACEUTICALS CO., LTD., Guangzhou (CN)

(21) Appl. No.: 18/831,145

(22) PCT Filed: Feb. 24, 2023
(86) PCT No.: PCT/CN2023/000042

§ 371 Date: Aug. 16, 2024

(30) Foreign Application Priority Data
Mar. 1, 2022 (CN) 202210196395.1

(51) Int. Cl. C07C 211/38; A61K 31/133; A61P 9/12

(52) U.S. Cl. CPC C07C 211/38; A61K 31/133; A61P 9/12; C07C 2603/74

(57) ABSTRACT

The present invention provides the use of an amantadine mononitrate compound in the manufacture of a medicament for the prevention and treatment of hypertension. The amantadine mononitrate compound can be administered to reduce the systolic and/or diastolic pressure of subjects. The amantadine mononitrate compound has a long half-life and long drug action time in subjects and can reduce the number of administration times and improve patient compliance as compared to existing hypertensive drugs. The medicament of the amantadine mononitrate compound of the present invention has no adverse reactions of orthostatic hypotension, and thus has improved the safety of the drug use.

### FIELD OF THE INVENTION

The present invention relates generally to the field of medicine and, more particularly, to the pharmaceutical use of amantadine mononitrate compounds, in particular to use of amantadine mononitrate compounds and pharmaceutically acceptable salts thereof in the preparation of medicaments for the prevention and treatment of hypertension.

### BACKGROUND OF THE INVENTION

Blood pressure refers to the pressure exerted on the walls of blood vessels when blood flows from the heart to various parts of the body. The human body has two circulatory systems, the pulmonary circulatory system and the systemic circulatory system. The pulmonary circulatory system is mainly the right heart circulatory system, which pumps venous blood through the right heart into the pulmonary artery, exchanges oxygen to obtain arterial blood, and then returns to the left heart, and then enters a systemic circulation state after entering the left heart, allowing oxygenated blood to be injected into the body through the left heart, thereby meeting the body's oxygen supply and energy needs. Unlike pulmonary hypertension, hypertension usually only refers to the cardiovascular syndrome with elevated systemic arterial pressure as the main clinical manifestation. Hypertension is the primary and modifiable risk factor leading to an increase in cardiovascular disease incidence and mortality among residents, with approximately 50% of cardiovascular disease incidence and 20% of cardiovascular disease deaths attributed to hypertension [Lancet. 2009 Nov. 21; 374(9703):1765-72]. According to the latest report on the Nutrition and Chronic Disease Status of Chinese Residents (2020), the prevalence of hypertension among adults in China is 27.5%, with a total population of approximately 300 million. It is estimated that the direct economic burden caused by hypertension exceeds 210.4 billion yuan. Up to 23.2% of adults in China have blood pressure levels of 130-139/80-89 mm Hg. Within 15 years, two-thirds of the middle-aged and young population will develop hypertension, and their risk of cardiovascular disease is 3.01 times that of those with blood pressure<130/80 mm Hg [Circulation. 2018 May 29; 137(22): 2344-2356]. If the blood pressure of adults in China is maintained at an ideal level (<130/80 mm Hg), it can prevent 44.1% of cardiovascular disease incidence [Sci China Life Sci 2018 May; 61 (5): 504-514]. Early prevention and treatment of hypertension in middle-aged and young people is crucial for reducing the long-term risk of cardiovascular disease [Cardiovascular Division of the Chinese Medical Association. Guidelines for Primary Prevention of Cardiovascular Disease in China [J]. Chinese Journal of Cardiovascular Disease, 2020, 48 (12): 1000-1038].

Hypertension is mainly characterized by an increase in systolic pressure and/or diastolic pressure. Systolic pressure (SP) is the lateral pressure exerted on the blood vessel wall by the blood injected from the ventricle into the artery when the heart contracts, at which point the blood pressure is at its maximum; at this time, the pressure on the inner wall is called systolic pressure, also known as high pressure. Diastolic pressure (DP) is of the end of diastole in the heart, when blood temporarily stops flowing into the artery, while the blood that flows into the artery continues to flow due to the elasticity and tension of the vascular wall, and there is still pressure on the vascular wall; at this time, the blood pressure is called diastolic pressure, also known as low pressure. For patients over the age of 50, systolic pressure is a more important and difficult to control cardiovascular disease risk factor than diastolic pressure, as it brings about rapid occurrence of cardiovascular and cerebrovascular accidents and does not have much reaction time. The damage to target organs (heart, brain, kidney, peripheral blood vessels, retina, etc.) caused by high systolic pressure in elderly people is more severe than that caused by elevated diastolic pressure. Therefore, conventional hypertension drugs such as calcium antagonists, β Receptor blockers, angiotensin I inhibitors, angiotensin receptor blockers, are mainly aimed at reducing systolic pressure. Guo Tingting from Beijing Union Medical College compared the effects of these hypertension drugs on 24-hour blood pressure in primary hypertension. The results showed that the calcium antagonist amlodipine had an average 24-hour dynamic systolic pressure decrease of 17.43 mmHg and an average 24-hour dynamic diastolic pressure decrease of 10.05 mmHg; The average 24-hour dynamic systolic pressure decreased by 6.74 mmHg and the average 24-hour dynamic diastolic pressure decreased by 3.33 mmHg with the angiotensin I inhibitor benazepril; The angiotensin receptor blocker valsartan resulted in an average 24-hour decrease in dynamic systolic pressure of 8.08 mmHg and an average 24-hour decrease in dynamic diastolic pressure of 6.04 mmHg; β receptor blocker metoprolol reduced the average 24-hour dynamic systolic pressure by 6.66 mmHg and the average 24-hour dynamic diastolic pressure by 4.6 mmHg; The average 24-hour dynamic systolic pressure decreased by 5.92 mmHg with tolasemide, and the average 24-hour dynamic diastolic pressure decreased by 2.42 mmHg (see Guo Tingting, Comparative study of the effects of six antihypertensive drugs on 24-hour blood pressure in primary hypertension [D]. Beijing Union Medical College, 2017). Although calcium antagonists β receptor blockers, angiotensin I inhibitors, angiotensin receptor blockers, and other commonly used hypertension drugs in clinical practice can also reduce diastolic pressure to a certain extent, but the decrease in diastolic pressure is significantly lower than that of systolic pressure.

In fact, high diastolic pressure is also a significant risk factor for cardiovascular and cerebrovascular diseases. A meta analysis conducted by the British Medical Research Council involving 1 million people showed that diastolic pressure is an important influencing factor for ischemic heart disease and stroke. With diastolic pressure at 75 mm Hg as the baseline, every 10 mm Hg increase in diastolic pressure doubles the risk of ischemic heart disease and stroke. The epidemiological research results of hypertension show that the majority of patients with elevated diastolic pressure account for 10% to 15% of all primary hypertension. Among middle-aged and young hypertensive individuals under the age of 40, 60% have simply elevated diastolic pressure [Chinese Journal of Hypertension, 2019, 27 (7): 615-621]. High diastolic pressure is more common in middle-aged and young people, but due to their good vascular elasticity, systolic pressure generally does not increase. If left uncontrolled for a long time, a simple increase in low pressure will transform into hypertension with both high and low pressures. Therefore, effectively reducing diastolic pressure can alleviate arteriosclerosis, while also delaying the course of hypertension and reducing the occurrence of related complications caused by continuous increase in blood pressure. Unfortunately, there are currently no specific therapeutic drugs for high diastolic pressure in clinical practice.

### SUMMARY OF THE INVENTION

To solve the problems in the prior art, a medical use of an amantadine mononitrate compound and a pharmaceutically acceptable salt thereof is provided in the present invention for the prevention and treatment of hypertension.

Therefore, the present invention provided use of an amantadine mononitrate compound and pharmaceutically acceptable salts thereof in the manufacture of a medicament for the prevention and treatment of hypertension; wherein the hypertension is hypertension with high diastolic pressure; and wherein the amantadine mononitrate compound has a structure of formula (I):

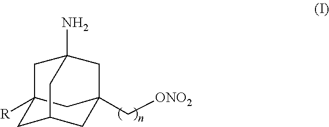

wherein, R is a straight-chain or branched-chain alkyl, preferably a C₁-C₆ alkyl.

The term “C₁-C₆ alkyl” as used herein refers to a straight-chain or branched-chain saturated hydrocarbon group with 1-6 carbon atoms, such as methyl, ethyl, propyl, isopropyl, n-butyl, isobutyl, sec-butyl, tert-butyl, n-pentyl, isopentyl, neopentyl, n-hexyl, or hexyl; preferably methyl, ethyl, propyl, isopropyl, n-butyl, or isobutyl; more preferably methyl, ethyl, propyl, or isopropyl.

Preferably, the amantadine mononitrate compound has a formula below:

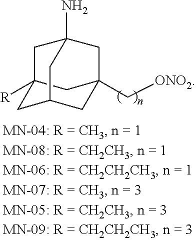

Further preferrably, the amantadine mononitrate compound is MN-08, which has the following formula:

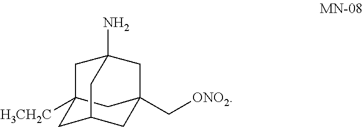

According to an embodiment of the invention, “a pharmaceutically acceptable salt” is a salt formed by a compound of formula I with an acid, such as hydrochloric acid (HCl), hydrobromic acid (HBr), hydroiodic acid, sulfuric acid, pyrosulfuric acid, phosphoric acid, nitric acid, methanesulfonic acid, ethanesulfonic acid, benzenesulfonic acid, p-toluenesulfonic acid, formic acid, acetic acid, acetoacetic acid, pyruvic acid, trifluoroacetic acid, propionic acid, butyric acid, hexanoic acid, heptanoic acid, undecanoic acid, Lauric acid, benzoic acid, salicylic acid, 2-(4-hydroxybenzoyl) benzoic acid, camphoric acid, cinnamic acid, cyclopentanepropanoic acid, digluconic acid, 3-hydroxy-2-naphthoic acid, nicotinic acid, pamoic acid, pectinic acid, persulfuric acid, 3-phenylpropanoic acid, picric acid, terpene acid, 2-hydroxyethanesulfonic acid, itaconic acid, amino sulfonic acid, trifluoromethanesulfonic acid, dodecyl sulfuric acid, 2-naphthalene sulfonic acid, naphthalene disulfonic acid, camphor sulfonic acid, citric acid, tartaric acid Stearic acid, lactic acid, oxalic acid, malonic acid, succinic acid, malic acid, fatty acid, algic acid, maleic acid, fumaric acid, D-gluconic acid, mandelic acid, ascorbic acid, glucoheptanoic acid, glycerol phosphate, aspartic acid, sulfosalicylic acid, homisulfuric acid, thiocyanic acid, 1,2-ethanedisulfonic acid, ethanolic acid, p-chlorobenzenesulfonic acid, trimethylacetic acid, tert-butyl acetic acid, hydroxynaphthalic acid, or glutamic acid.

It should be noted that pulmonary hypertension is not a hypertension. The hypertension described herein refers only to systemic hypertension, whereas pulmonary hypertension is a hypertension of pulmonary circulation but not the hypertension commonly understood in the art.

According to some embodiments of the invention, the hypertension is hypertension with high diastolic pressure, including but not limited to DP≥80 mmHg; For example, DP of 80-89 mmHg, or DP of 90-99 mmHg, or DP of 100-109 mmHg, or DP≥110 mmHg.

As provided in the present invention, in the use of an amantadine mononitrate compound and pharmaceutically acceptable salts thereof in the manufacture of a medicament for the prevention and treatment of hypertension, the hypertension refers to hypertension with high diastolic pressure, which may include hypertension with simple high diastolic pressure. There is no special requirement for systolic pressure (SP), and the systolic pressure can be high, for example, ≥130 mmHg, such as 130-139 mmHg, 140-159 mmHg, 160-179 mmHg, or ≥180 mmHg; The systolic pressure can also be low, for example, <130 mmHg. These are all within the scope of protection of the present invention.

The hypertension as described herein can be defined as the hypertension based on the hypertension guidelines prepared jointly by the American Heart Association/American Heart Association in 2017, with SP/DP≥130/80 mm Hg (2017 ACC/AHA/AAPA/ABC/ACPM/AGS/APhA/ASH/ASPC/NMA/PCNA Guideline for the Prevention, Detection, Evaluation, and Management of High Blood Pressure in Adults: A Report of the American College of Cardiology/American Heart Association Task Force on Clinical Practice Guidelines).

The hypertension as described herein can be also defined according to Chinese Guidelines for the Prevention and Treatment of Hypertension (2018 Revision) to classify blood pressure as normal blood pressure (SP<120 mmHg and DP<80 mmHg), normal high value (SP 120-139 mmHg and/or DP 80-89 mmHg), and hypertension (SP≥140 mmHg and/or DP≥90 mmHg). Hypertension refers to the measurement of blood pressure in the clinic three times on different days without the use of antihypertensive drugs, with SP 140 mmHg and/or DP≥90 mmHg. Wherein, SP 140 mmHg and DP<90 mmHg are of simple high systolic pressure; and SP<140 mmHg and DP≥90 mmHg are of simple high diastolic pressure. A patient who has a history of hypertension and is currently using antihypertensive drugs, although having a blood pressure below 140/90 mmHg, should still be diagnosed with hypertension. Based on the level of elevated blood pressure, hypertension is further divided into grades 1, 2, and 3: grade 1 hypertension (mild), SP 140-159 mmHg and/or DP 90-99 mmHg; grade 2 hypertension (moderate), SP 160-179 mmHg and/or DP 100-109 mmHg; grade 3 hypertension (severe), SP≥180 mmHg and/or DP≥110 mmHg.

The present invention has the following advantages over the prior art technologies:
(1) Previous studies (CN 106344551 A) found that amantadine mononitrate compounds can reduce pulmonary hypertension, and animal experiments showed that amantadine mononitrate compounds can slightly increase normal rat cerebral blood flow and have a certain increase in peripheral blood pressure. In the clinical trial of the amantadine mononitrate compound MN-08, it was unexpectedly found that the amantadine mononitrate compounds of the present invention can reduce the systolic pressure and/or diastolic pressure of subjects, and the diastolic pressure decreases more than the systolic pressure. This is of great significance for the treatment or prevention of hypertension with high diastolic pressure in young and middle-aged people.
(2) In the clinical trial of the amantadine mononitrate compound MN-08, both the single dose group and the multiple dose group showed varying degrees of decrease in blood pressure.
(3) The amino adamantane mononitrate compound MN-08 has a long half-life and drug action time in subjects, which can reduce the number of administration times and improve patient compliance as compared to existing hypertensive drugs.

### DETAILED DESCRIPTION OF THE EMBODIMENTS

The following embodiments provide a detailed description of the technologies of the present invention. However, it should be understood that these specific embodiments are only for illustration and not to limit the scope of the present invention.

All subjects in the following embodiments have signed informed consent forms, which comply with medical ethics regulations. According to the relevant provisions of Chinese Guidelines for the Prevention and Treatment of Hypertension (2018 Revision), the values measured for hypertension are the average blood pressure measured three times on a different day without the use of antihypertensive drugs. Therefore, in this embodiment, the baseline blood pressure of the subjects before administration is set as the average blood pressure measured at three different times: screening period, occupancy period, and 1 hour before administration, as shown in Tables 1 to 3.

### Example 1: Pharmacokinetic Test of MN-08

In the clinical trial of amantadine mononitrate compound MN-08, the pharmacokinetic test included a total of 8 dose groups (6 mg, 12 mg, 42 mg, 60 mg, 78 mg, 96 mg, 114 mg, and 132 mg).

The subjects received a single oral dose of MN-08 at doses of 6 mg, 12 mg, 42 mg, 60 mg, 78 mg, 96 mg, 114 mg, and 132 mg, respectively. The pharmacokinetic results showed that after administering different doses of MN-08 to each group of subjects, the peak concentration was reached at 2.5-6.5 hours. In the dose range of 6 mg to 132 mg, the half-life t_{1/2} of oral administration of MN-08 ranges from 20 hours to 30 hours, and t_{1/2} extends with increasing dosage. Most existing antihypertensive drugs have a short half-life and require frequent administration, resulting in poor patient compliance. In contrast, the amantadine mononitrate compound MN-08 of the present invention has a half-life of more than 20 hours, a long drug action time, and can reduce the number of administration times and improve patient compliance compared to other existing hypertensive drugs.

### Example 2: Effect of MN-08 on Subjects' Blood Pressure

In the clinical trial of amantadine mononitrate compound MN-08, it is investigated the effect of MN-08 on blood pressure in subjects. The experiments were carried out to analyze the blood pressure status of subjects in both the single dose and multiple dose groups.

### (1) Single Dose Group

During a single administration, each dose group is required to fast for at least 10 hours before administration, without water deprivation overnight, and on the day of administration, the medication is taken on an empty stomach at around 8 a.m., with 240 mL of water administered. Do not drink for 1 hour before and 1 hour after taking the medication, do not eat for 4 hours, and maintain upper body upright for 2 hours after taking the medication (except when lying flat during electrocardiogram testing). The experimental results show that a single dose of MN-08 can reduce the blood pressure of subjects in all dose groups (6 mg, 12 mg, 42 mg, 60 mg, 78 mg, 96 mg, 114 mg, and 132 mg), especially in terms of diastolic pressure. Even for the subjects with the lowest dose of 6 mg in the experiment, the systolic pressure at 6 hours can be reduced by 13.2%, while the diastolic pressure at 6 hours can be reduced by 17.1%, with a greater decrease in diastolic pressure than systolic pressure. The effect of a single administration of 12-132 mg of MN-08 on the blood pressure of subjects is shown in Table 1 below.

TABLE 1
Effect of single administration of MN-08 on blood pressure of subjects

| | | | | Inspection | SP | SP (reduce | DP | DP (reduce |
|---|---|---|---|---|---|---|---|---|
| Subject | Dose | | Visit | time point | (mmHg) | amplitude) | (mmHg) | amplitude) |
| Subject 1 | 12 | mg | Baseline b.p. | | 122 | | 77 | |
| | | | before adm. | | | | | |
| Subject 1 | 12 | mg | Single dose gr. | 6 hrs after | 104 | −14.8% | 64 | −16.9% |
| | | | (D 1) | adm. | | | | |
| Subject 1 | 12 | mg | Single dose gr. | 12 hrs after | 109 | −10.7% | 60 | −22.1% |
| | | | (D 1) | adm. | | | | |
| Subject 2 | 42 | mg | Baseline b.p. | | 113 | | 79 | |
| | | | before adm. | | | | | |
| Subject 2 | 42 | mg | Single dose gr. | 6 hrs after | 96 | −15.0% | 63 | −20.3% |
| | | | (D 1) | adm. | | | | |
| Subject 2 | 42 | mg | Single dose gr. | 12 hrs after | 96 | −15.0% | 69 | −12.7% |
| | | | (D 1) | adm. | | | | |
| Subject 3 | 42 | mg | Baseline b.p. | | 124 | | 85 | |
| | | | before adm. | | | | | |
| Subject 3 | 42 | mg | Single dose gr. | 6 hrs after | 118 | −4.8% | 72 | −15.3% |
| | | | (D 1) | adm. | | | | |
| Subject 3 | 42 | mg | single dose gr. | 12 hrs after | 98 | −21.0% | 61 | −28.2% |
| | | | (D 1) | adm. | | | | |
| Subject 4 | 60 | mg | Baseline b.p. | | 107 | | 78 | |
| | | | before adm. | | | | | |
| Subject 4 | 60 | mg | Single dose gr. | 6 hrs after | 93 | −13.1% | 58 | −25.6% |
| | | | (D 1) | adm. | | | | |
| Subject 4 | 60 | mg | Single dose gr. | 12 hrs after | 90 | −15.9% | 58 | −25.6% |
| | | | (D 1) | adm. | | | | |
| Subject 5 | 60 | mg | Baseline b.p. | | 111 | | 73 | |
| | | | before adm. | | | | | |
| Subject 5 | 60 | mg | Single dose gr. | 6 hrs after | 93 | −16.2% | 62 | −15.1% |
| | | | (D 1) | adm. | | | | |
| Subject 5 | 60 | mg | Single dose gr. | 12 hrs after | 95 | −14.4% | 61 | −16.4% |
| | | | (D 1) | adm. | | | | |
| Subject 6 | 78 | mg | Baseline b.p. | | 113 | | 67 | |
| | | | before adm. | | | | | |
| Subject 6 | 78 | mg | Single dose gr. | 6 hrs after | 103 | −8.8% | 61 | −9.0% |
| | | | (D 1) | adm. | | | | |
| Subject 6 | 78 | mg | Single dose gr. | 12 hrs after | 104 | −8.0% | 54 | −19.4% |
| | | | (D 1) | adm. | | | | |
| Subject 7 | 78 | mg | Baseline b.p. | | 106 | | 74 | |
| | | | before adm. | | | | | |
| Subject 7 | 78 | mg | Single dose gr. | 6 hrs after | 94 | −11.3% | 62 | −16.2% |
| | | | (D 1) | adm. | | | | |
| Subject 7 | 78 | mg | Single dose gr. | 12 hrs after | 102 | −3.8% | 60 | −18.9% |
| | | | (D 1) | adm. | | | | |
| Subject 8 | 96 | mg | Baseline b.p. | | 101 | | 69 | |
| | | | before adm. | | | | | |
| Subject 8 | 96 | mg | Single dose gr. | 6 hrs after | 79 | −21.8% | 51 | −26.1% |
| | | | (D 1) | adm. | | | | |
| Subject 8 | 96 | mg | Single dose gr. | 12 hrs after | 87 | −13.8% | 61 | −11.6% |
| | | | (D 1) | adm. | | | | |
| Subject 9 | 96 | mg | Baseline b.p. | | 121 | | 78 | |
| | | | before adm. | | | | | |
| Subject 9 | 96 | mg | Single dose gr. | 6 hrs after | 105 | −13.2% | 63 | −19.2% |
| | | | (D 1) | adm. | | | | |
| Subject 9 | 96 | mg | Single dose gr. | 12 hrs after | 103 | −14.8% | 68 | −12.8% |
| | | | (D 1) | adm. | | | | |
| Subject 10 | 114 | mg | Baseline b.p. | | 115 | | 75 | |
| | | | before adm. | | | | | |
| Subject 10 | 114 | mg | Single dose gr. | 6 hrs after | 92 | −20.0% | 62 | −17.3% |
| | | | (D 1) | adm. | | | | |
| Subject 10 | 114 | mg | Single dose gr. | 12 hrs after | 90 | −21.7% | 57 | −24.0% |
| | | | (D 1) | adm. | | | | |
| Subject 11 | 114 | mg | Baseline b.p. | | 126 | | 85 | |
| | | | before adm. | | | | | |
| Subject 11 | 114 | mg | Single dose gr. | 6 hrs after | 119 | −5.6% | 68 | −20.0% |
| | | | (D 1) | adm. | | | | |
| Subject 11 | 114 | mg | Single dose gr. | 12 hrs after | 105 | −16.7% | 68 | −20.0% |
| | | | (D 1) | adm. | | | | |
| Subject 12 | 132 | mg | Baseline b.p. | | 123 | | 78 | |
| | | | before adm. | | | | | |
| Subject 12 | 132 | mg | Single dose gr. | 6 hrs after | 120 | −2.4% | 59 | −24.4% |
| | | | (D 1) | adm. | | | | |
| Subject 12 | 132 | mg | Single dose gr. | 12 hrs after | 105 | −14.6% | 60 | −23.1% |
| | | | (D 1) | adm. | | | | |
| Subject 13 | 132 | mg | Baseline b.p. | | 106 | | 68 | |
| | | | before adm. | | | | | |
| Subject 13 | 132 | mg | Single dose gr. | 6 hrs after | 97 | −8.5% | 54 | −20.6% |
| | | | (D 1) | adm. | | | | |
| Subject 13 | 132 | mg | Single dose gr. | 12 hrs after | 96 | −9.4% | 56 | −17.6% |

| | |
|---|---|
| (D 1) | adm. |

### (2) Multiple Administration Group

Multiple dosing trials: Participants in each dose group fasted for at least 10 hours before administration, without water deprivation overnight. Each group of subjects received oral administration of the test drug on an empty stomach from day 1 to day 6, twice a day (with an interval of 12 hours and at least 1 hour on an empty stomach before the second administration), and only once in the morning on day 7. A total of 13 doses were administered. The first administration time every day is around 8 am, taken with 240 mL of water. Taking subject 14 as an example, the effect of multiple doses of MN-08 (7 days) on subject blood pressure was investigated. The data is shown in Table 2.

TABLE 2
Effect of Multiple Administrations of MN-08 on Blood Pressure of Subjects (7 Days)

| | | | Inspection | SP | DP |
|---|---|---|---|---|---|
| Subject | Dose | Visit | time point | (mmHg) | (mmHg) |
| Subject 14 | 12 mg | Baseline b.p. before adm. | | 122 | 84 |
| Subject 14 | 12 mg | Multiple adm. gr. (D 1) | 4 hrs after 1st adm. | 122 | 81 |
| Subject 14 | 12 mg | Multiple adm. gr. (D 1) | 8 hrs after 1st adm. | 114 | 74 |
| Subject 14 | 12 mg | Multiple adm. gr. (D 1) | 2 hrs after 2nd adm. | 113 | 73 |
| Subject 14 | 12 mg | Multiple adm. gr. (D 2) | 4 hrs after 1st adm. | 115 | 76 |
| Subject 14 | 12 mg | Multiple adm. gr. (D 2) | 8 hrs after 1st adm. | 117 | 68 |
| Subject 14 | 12 mg | Multiple adm. gr. (D 2) | 2 hrs after 2nd adm. | 107 | 77 |
| Subject 14 | 12 mg | Multiple adm. gr. (D 3) | 4 hrs after 1st adm. | 114 | 74 |
| Subject 14 | 12 mg | Multiple adm. gr. (D 3) | 8 hrs after 1st adm. | 111 | 82 |
| Subject 14 | 12 mg | Multiple adm. gr. (D 3) | 2 hrs after 2nd adm. | 109 | 73 |
| Subject 14 | 12 mg | Multiple adm. gr. (D 4) | 4 hrs after 1st adm. | 109 | 73 |
| Subject 14 | 12 mg | Multiple adm. gr. (D 4) | 8 hrs after 1st adm. | 107 | 70 |
| Subject 14 | 12 mg | Multiple adm. gr. (D 4) | 2 hrs after 2nd adm. | 109 | 73 |
| Subject 14 | 12 mg | Multiple adm. gr. (D 5) | 4 hrs after 1st adm. | 106 | 69 |
| Subject 14 | 12 mg | Multiple adm. gr. (D 5) | 8 hrs after 1st adm. | 97 | 62 |
| Subject 14 | 12 mg | Multiple adm. gr. (D 5) | 2 hrs after 2nd adm. | 114 | 74 |
| Subject 14 | 12 mg | Multiple adm. gr. (D 6) | 4 hrs after 1st adm. | 118 | 77 |
| Subject 14 | 12 mg | Multiple adm. gr. (D 6) | 8 hrs after 1st adm. | 112 | 63 |
| Subject 14 | 12 mg | Multiple adm. gr. (D 6) | 2 hrs after 2nd adm. | 125 | 77 |
| Subject 14 | 12 mg | Multiple adm. gr. (D 7) | 4 hrs after adm. | 108 | 77 |
| Subject 14 | 12 mg | Multiple adm. gr. (D 7) | 8 hrs after adm. | 102 | 62 |
| Subject 14 | 12 mg | Multiple adm. gr. (D 7) | 12 hrs after adm. | 103 | 62 |

Table 2 shows that MN-08 was continuously administered twice a day for 7 days, and both systolic and diastolic pressure of the subjects were reduced. Overall, the decrease in diastolic pressure is greater than that in systolic pressure.

Multiple dosing trials: Participants in each dose group fasted for at least 10 hours before administration, without water deprivation overnight. Each group of subjects took the test drug orally on an empty stomach twice a day (with an interval of 12 hours and at least 1 hour on an empty stomach before the second administration). The first administration time every day is around 8 am, taken with 240 mL of water. Taking subjects 15-17 as an example, the effect of multiple doses of MN-08 (3 days) on subjects' blood pressure was investigated. The data is shown in Table 3.

TABLE 3
Effect of multiple administration of MN-08 on blood pressure of subjects (3 days)

| | | | Inspection | SP | DP |
|---|---|---|---|---|---|
| Subject | Dose | Visit | time point | (mmHg) | (mmHg) |
| Subject 15 | 12 mg | Baseline b.p. before adm. | | 108 | 75 |
| Subject 15 | 12 mg | Multiple adm. gr. (D 1) | 4 hrs after 1st adm. | 96 | 61 |
| Subject 15 | 12 mg | Multiple adm. gr. (D 1) | 8 hrs after 1st adm. | 100 | 51 |
| Subject 15 | 12 mg | Multiple adm. gr. (D 1) | 2 hrs after 2nd adm. | 94 | 59 |
| Subject 15 | 12 mg | Multiple adm. gr. (D 2) | 4 hrs after 1st adm. | 92 | 56 |
| Subject 15 | 12 mg | Multiple adm. gr. (D 2) | 8 hrs after 1st adm. | 108 | 57 |
| Subject 15 | 12 mg | Multiple adm. gr. (D 2) | 2 hrs after 2nd adm. | 95 | 64 |
| Subject 15 | 12 mg | Multiple adm. gr. (D 3) | 4 hrs after 1st adm. | 95 | 64 |
| Subject 15 | 12 mg | Multiple adm. gr. (D 3) | 8 hrs after 1st adm. | 86 | 57 |
| Subject 15 | 12 mg | Multiple adm. gr. (D 3) | 2 hrs after 2nd adm. | 105 | 65 |
| Subject 16 | 24 mg | Baseline b.p. before adm. | | 118 | 81 |
| Subject 16 | 24 mg | Multiple adm. gr.(D 1) | 4 hrs after 1st adm. | 97 | 61 |
| Subject 16 | 24 mg | Multiple adm. gr.(D 1) | 8 hrs after 1st adm. | 102 | 63 |
| Subject 16 | 24 mg | Multiple adm. gr.(D 1) | 2 hrs after 2nd adm. | 103 | 66 |
| Subject 16 | 24 mg | Multiple adm. gr.(D 2) | 4 hrs after 1st adm. | 106 | 69 |
| Subject 16 | 24 mg | Multiple adm. gr.(D 2) | 8 hrs after 1st adm. | 99 | 65 |
| Subject 16 | 24 mg | Multiple adm. gr.(D 2) | 2 hrs after 2nd adm. | 100 | 61 |
| Subject 16 | 24 mg | Multiple adm. gr.(D 3) | 4 hrs after 1st adm. | 107 | 67 |
| Subject 16 | 24 mg | Multiple adm. gr.(D 3) | 8 hrs after 1st adm. | 95 | 63 |
| Subject 16 | 24 mg | Multiple adm. gr.(D 3) | 2 hrs after 2nd adm. | 98 | 69 |
| Subject 17 | 24 mg | Baseline b.p. before adm. | | 126 | 87 |
| Subject 17 | 24 mg | Multiple adm. gr.(D 1) | 4 hrs after 1st adm. | 126 | 81 |
| Subject 17 | 24 mg | Multiple adm. gr.(D 1) | 8 hrs after 1st adm. | 112 | 66 |
| Subject 17 | 24 mg | Multiple adm. gr.(D 1) | 2 hrs after 2nd adm. | 107 | 73 |
| Subject 17 | 24 mg | Multiple adm. gr.(D 2) | 4 hrs after 1st adm. | 102 | 73 |
| Subject 17 | 24 mg | Multiple adm. gr.(D 2) | 8 hrs after 1st adm. | 115 | 68 |
| Subject 17 | 24 mg | Multiple adm. gr.(D 2) | 2 hrs after 2nd adm. | 94 | 63 |
| Subject 17 | 24 mg | Multiple adm. gr.(D 3) | 4 hrs after 1st adm. | 107 | 70 |
| Subject 17 | 24 mg | Multiple adm. gr.(D 3) | 8 hrs after 1st adm. | 111 | 76 |
| [symbol] 17 | 24 mg | Multiple adm. gr.(D 3) | 2 hrs after 2nd adm. | 110 | 74 |

Table 3 shows that continuous administration of MN-08 for 3 days, twice a day, and each dose group (12 mg, 24 mg) can lower the blood pressure of the subjects. Overall, the decrease in diastolic pressure is greater than that in systolic pressure.

According to the 2017 Guidelines on Hypertension jointly developed by the American College of Cardiology/American Heart Association, SP/DP≥130/80 mm Hg is defined as hypertension. According to this standard, many participants in clinical trials of the amantadine mononitrate compound MN-08 belong to patients with grade 1 hypertension. From the clinical trial results, it can be seen that the amino amantadine mononitrate compound MN-08 can significantly reduce the systolic and/or diastolic pressure of subjects, with a greater decrease in diastolic pressure than systolic pressure.

From this, it can be seen that making diastolic pressure drop more than systolic pressure drop is the characteristic of MN-08. The inventor followed the experimental method of single or multiple administration as described in Example 2 to investigate the effect of MN-08 on blood pressure in subjects with a DP≥90 mmHg, which can also demonstrate its significant effect on reducing diastolic pressure.

1. Use of an amantadine mononitrate compound and a pharmaceutically acceptable salt thereof in manufacture of a medicament, wherein the medicament comprising the amantadine mononitrate compound or a pharmaceutically acceptable salt thereof for the prevention and treatment of hypertension; wherein the hypertension is hypertension with high diastolic pressure; and wherein the amantadine mononitrate compound has a structure of formula (I): 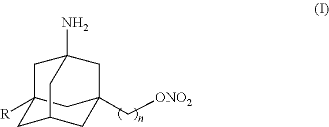
wherein, R is a straight-chain or branched-chain alkyl, and n is 1-6.

2. (canceled)

3. The use of claim 1, wherein the amantadine mononitrate compound has a formula below:

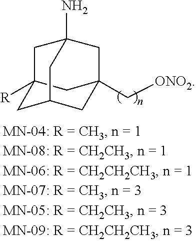

4. The use of claim 4, wherein the amino amantadine mononitrate compound is MN-08 having the following formula:

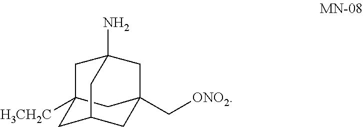

5. The use of claim 1, wherein the pharmaceutically acceptable salt is a salt formed by the amino amantadine mononitrate compound and an acid; wherein the acid is selected from: hydrochloric acid (HCl), hydrobromic acid (HBr), hydroiodic acid, sulfuric acid, pyrosulfuric acid, phosphoric acid, nitric acid, methanesulfonic acid, ethanesulfonic acid, benzenesulfonic acid, p-toluenesulfonic acid, formic acid, acetic acid, acetoacetic acid, pyruvic acid, trifluoroacetic acid, propionic acid, butyric acid, hexanoic acid, heptanoic acid, undecanoic acid, Lauric acid, benzoic acid, salicylic acid, 2-(4-hydroxybenzoyl) benzoic acid, camphoric acid, cinnamic acid, cyclopentanepropanoic acid, digluconic acid, 3-hydroxy-2-naphthoic acid, nicotinic acid, pamoic acid, pectinic acid, persulfuric acid, 3-phenylpropanoic acid, picric acid, terpene acid, 2-hydroxyethanesulfonic acid, itaconic acid, amino sulfonic acid, trifluoromethanesulfonic acid, dodecyl sulfuric acid, 2-naphthalene sulfonic acid, naphthalene disulfonic acid, camphor sulfonic acid, citric acid, tartaric acid Stearic acid, lactic acid, oxalic acid, malonic acid, succinic acid, malic acid, fatty acid, algic acid, maleic acid, fumaric acid, D-gluconic acid, mandelic acid, ascorbic acid, glucoheptanoic acid, glycerol phosphate, aspartic acid, sulfosalicylic acid, hemisulfuric acid, thiocyanic acid, 1,2-ethanedisulfonic acid, ethanolic acid, p-chlorobenzenesulfonic acid, trimethylacetic acid, tert-butyl acetic acid, hydroxynaphthalic acid, or glutamic acid.

6. The use of claim 1, wherein the diastolic pressure is ≥80 mmHg.

7. The use of claim 6, wherein the diastolic pressure is 80 to 89 mmHg.

8. The use of claim 7, wherein the diastolic pressure is 90 to 99 mmHg.

9. The use of claim 6, wherein the diastolic pressure is 100 to 109 mmHg.

10. The use of claim 6, wherein the diastolic pressure is ≥110 mmHg.

11. The use of claim 1, wherein the medicament further comprises a pharmaceutically acceptable carrier.
